(19) United States
(12) Patent Application Publication
(10) Pub. No.: US 2009/0202461 A1
(43) Pub. Date: Aug. 13, 2009

(54) Anhydrous cosmetic composition comprising a crosslinked polyrotaxane and an oil

(76) Inventors: Ivan Rodriguez, Cauffry (FR); Timo Luukas, Massy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/920,229

(22) PCT Filed: Jun. 6, 2006
(86) PCT No.: PCT/EP2006/006155

§ 371 Date: Sep. 11, 2008

Related U.S. Application Data
(60) Provisional application No. 60/690,883, filed on Jun. 16, 2005.

(30) Foreign Application Priority Data
Jun. 10, 2005 (FR) 0551569

(51) Int. Cl. A61K 8/72; A61K 8/73; A61K 8/81; A61K 8/88

(52) U.S. Cl. 424/63; 424/70.13; 424/70.15; 424/70.16; 424/70.17; 424/70.11; 424/78.03; 424/70.12

(57) ABSTRACT

The invention relates to an anhydrous cosmetic composition intended to be applied to keratinous substances, in particular the skin, the lips or the eyelashes. This composition comprises at least one crosslinked polyrotaxane polymer and at least one oil. This composition makes it possible in particular to form, on keratinous substances, a deposited layer which increases in volume by addition of water or by the action of saliva or sweat. This composition is in particular a mascara, a lipstick or a foundation formulation.

The present invention relates to an anhydrous cosmetic composition intended to be applied to keratinous substances, in particular the skin, lips or eyelashes, comprising a crosslinked polyrotaxane.

The said composition absorbs water once the composition has been applied to keratinous substances, thus bringing about an increase in volume of its deposited layer. The keratinous substances thus give the impression of being thicker, fuller or smoother by filling in their rough edges.

Consumers are looking for cosmetic products which make it possible to obtain an increase in the perception of the volume of the keratinous substances which they desire to make up. In particular, a loading effect on the eyelashes is desired for mascaras, a fullness effect is desired for glosses and lipsticks, modelling properties, and properties of masking imperfections of the skin are required by users of foundations.

It is an object of the present invention to provide cosmetic products capable of forming, on keratinous substances, a deposited layer having a volume which does not decrease over time, in contrast to the deposited layer of a conventional cosmetic composition. In particular, the deposited layer of the composition is capable of increasing in volume over time after application to keratinous substances by the action of an external stimulus, such as atmospheric moisture, sweat, saliva or a lotion applied to the said deposited layer capable of forming a gel in the presence of the crosslinked polyrotaxane.

The composition according to the invention additionally exhibits satisfactory mechanical or rheological properties, such as a pleasant texture, suited to the use of the composition, and a satisfactory elasticity.

The present invention is targeted at providing compositions which make it possible to obtain cosmetic compositions which create an optical effect of volume once applied to a substrate, such as the skin, lips or superficial body growths. These compositions, after application, for example, to the cheeks, eyelids, eyelashes or lips, provide a perception of the volume which is different from that of the non-made-up substrate.

The application to keratinous substances of a known cosmetic composition results in a deposited layer which can become thinner over time, by penetration of all or part of the composition into the keratinous substances and/or by evaporation of the volatile constituents initially present in the deposited layer.

These phenomena may then reveal defects of the substrate, such as fine lines, defects of pigmentation, such as blemishes on the hands and face, loss in colour of the lips, or rosacea, which is particularly troublesome for a user of foundation. This is because one of the aims of a foundation is to conceal imperfections (blemishes, blackheads) of the skin and to give the skin a uniform complexion.

The document EP 1 069 151 discloses a cosmetic composition comprising at least one organic polymer, at least one first volatile solvent incompatible with the organic polymer and a second non-volatile solvent compatible with the organic polymer. The volatile and non-volatile solvents are such that they make possible the swelling of the polymer by dissolution of the latter in the non-volatile solvents as the volatile solvents evaporate from the substrate on which the composition is deposited. As the volatile solvents evaporate, the polymer changes from the insoluble state to the soluble state and forms a network of entanglements which traps the non-volatile solvents, thus bringing about an increase in the volume of the deposited layer of the composition on the keratinous substances.

Consumers are also looking for compositions which make it possible to remodel the face, in particular to enhance the cheekbones and/or to render the lips full. Currently, the increase in volume of certain parts of the face or body is obtained by injection of substances such as silicone gels. This type of remodelling is generally carried out under local anaesthesia. In addition, this type of remodelling is lengthy, tedious and expensive.

Furthermore, it is known that an effect of volume can be produced by applying a light tint and a dark tint side by side, the light tint being applied to the area which it is desired to enhance. To obtain this effect conventionally requires the use of two different compositions and depends on the skill of the person who is applying them. Furthermore, this technique is difficult to carry out in making up the lips.

The use of optical effect pigments, such as goniochromatic pigments, has been disclosed for modifying the perception of the volume of the part of the body to which the composition is applied, according to the angle of observation or the angle of incidence of the light.

Thus, Application EP 0 953 330 discloses a make-up kit combining a first goniochromatic pigment and a second pigment having one of the colours of the first pigment. International Application WO 01/51015 provides compositions which combine, with conventional interference pigments, a four-layer interference pigment, also known as shadow pigment, which exhibits a luminosity which can vary according to the angle of specular reflection.

Application EP 1 382 323 discloses a composition comprising at least one goniochromatic colouring agent capable of creating a goniochromatic coloured background and reflective particles capable of creating highlight points visible to the naked eye.

Compositions for making up keratinous fibres, in particular the eyelashes, generally have a high solids content in order to contribute material to the keratinous fibres and must obtain a make-up result in which volume or loading are more or less bestowed.

Nevertheless, the increase in the solids content of solids such as waxes, fillers or pigments leads to an increase in the consistency of the product obtained and thus to an application to fibres which is problematic and difficult as the composition is thick and viscous, gives a granular and non-smooth appearance to the deposited layer and is deposited with difficulty, in heterogeneous fashion and in clusters.

Another means for improving the body-bestowing effect is to increase the adhesion of the composition to the keratinous fibres in order to promote attachment when it is applied. For this, use is made of tackifying additives, which, however, cannot be incorporated at high levels for reasons of feasibility, as they render the composition compact, and for reasons of cosmetic quality, as they become excessively tacky on application.

Yet another means for providing a loading effect to mascara is to add a polymer which can be stimulated by heat, for example an Expancel, such as disclosed in Application EP 1 525 876. However, this polymer requires a very high activation temperature for swelling.

It is an object of the present invention to provide a novel route for the formulation of a cosmetic composition capable of generating a thick deposited layer on keratinous substances while exhibiting satisfactory mechanical and rheological properties.

A subject-matter of the invention is an anhydrous cosmetic composition comprising at least one crosslinked polyrotaxane and at least one oil.

Polyrotaxanes form part of the chemical family of the inclusion compounds, which comprise a first molecular entity which forms a cavity of limited size in which is housed a molecular entity of a second chemical type.

JP09216815 of Noevir Co. Ltd (1997) and JP09315937 of Shiseido Co. Ltd (1997) disclose cosmetic products comprising pseudopolyrotaxanes. These molecules comprise a backbone on which are included cyclic molecules (cyclodextrins). However, the compositions do not increase in volume sufficiently once applied to keratinous substances and their hold over time is low. In addition, the cyclic molecules have a tendency to become unstrung when the pseudopolyrotaxane is dissolved.

The Applicant has found, surprisingly, that the formulation of a specific polyrotaxane in an anhydrous cosmetic medium makes possible a deposited layer on keratinous substances, the volume of which is capable of increasing by the action of an external stimulus, making it possible to lastingly conceal defects of appearance of keratinous substances (blemishes, shadows under the eyes, folds, hollows, thinness) and making it possible to confer an increased volume on the eyelashes or lips. The moistening of the deposited layer can take place by addition of water, prior or subsequent to the deposition of the composition, or by the action of saliva, sweat or ambient moisture.

The term “anhydrous composition” is understood to mean a composition comprising less than 5% by weight of water, more preferably less than 2% by weight of water. According to one embodiment, the water possibly present in the composition is not added to the ingredients during its preparation. This water may be bonded in the form of trace amounts to the ingredients used to prepare the said composition.

The composition according to the invention is such that it is capable of forming a gel once applied to keratinous substances. The polyrotaxane is capable of increasing in volume by formation of a gel in the presence of a fluid. This fluid, preferably a hydrophilic fluid, can be, for example, sweat, saliva, tears or any other natural or artificial liquid, such as water, a water-in-oil emulsion or a lotion, capable of being deposited on the substrate to be made up, before or after application of a cosmetic composition in accordance with the invention.

### Crosslinked Polyrotaxane

The term “pseudopolyrotaxane” is understood to mean a supermolecular edifice which comprises at least one linear molecule and at least two cyclic molecules strung along the said linear molecule, the linear molecule and the cyclic molecules not being bonded via covalent bonds, with the result that the cyclic molecules can freely move along the linear molecule.

A “polyrotaxane” is obtained from a pseudopolyrotaxane, to which is attached, at each end of the linear molecule, a molecular structure which prevents the cyclic molecules and the linear molecule from separating, if appropriate.

The term “crosslinked polyrotaxane” is understood to mean a compound comprising at least one first polyrotaxane and one second polyrotaxane, at least one cyclic molecule of the first polyrotaxane and at least one cyclic molecule of the second polyrotaxane being bonded via at least one bond which can be chemical or physical. The bond can in particular be a metallic bond, an ionic bond, a covalent bond, an interaction resulting from the formation of charge transfer complexes, a weak interaction of hydrogen bond, Van der Waal's bond or π-π bond type, or a mixture of these.

A polyrotaxane is thus a supermolecular assemblage in which cyclic molecules are “included” by a linear molecule. To prevent the cyclic molecules from becoming unstrung from the linear molecule, the ends of the linear molecule are functionalized by bulky or ionic groups.

### Linear Molecules

In the present invention, the expression “linear molecule” is intended to denote a substantially “linear” molecule. This means that a linear molecule can comprise one or more branch chains, provided that the cyclic molecules can be rotated about or moved along the linear molecule.

The length of the “linear” molecule is not limited to a specific length, provided that the linear molecule allows the cyclic molecules to turn round on themselves or to move along the said linear molecule.

The linear molecules can be chosen from polymers, in particular:
hydrophilic polymers, such as a poly(vinyl alcohol), a polyvinylpyrrolidone, a poly((meth)acrylic acid), polymers derived from cellulose (carboxymethyl-cellulose, hydroxyethylcellulose, hydroxypropyl-cellulose and the like), a polyacrylamide, polyalkylene glycols, such as polyethylene glycols and polypropylene glycols, polytetrahydrofurans, poly(vinyl acetal)s, a poly(vinyl methyl ether), polyamines, polyethylene-imine, casein, gelatin, starch, and their copolymers;
hydrophobic polymers, for example polyolefins, such as polyethylenes, polypropylenes, polyisoprenes, polyisobutylenes or polybutadienes; copolymers of olefins, such as ethylene/butylene copolymers; poly-esters, polydimethylsiloxanes, poly(vinyl chloride), polystyrene, acrylonitrile/styrene copolymers, polymers and copolymers of (meth)acrylic esters, such as poly(methyl methacrylate) or acrylonitrile/methyl acrylate copolymers; polycarbonates, polyurethanes, vinyl chloride/vinyl acetate copolymers or poly(vinyl butyral);
   and their derivatives.

Preference is given, among these compounds, to polyethylene glycols, polyisoprenes, polyisobutylenes, polybutadienes, polypropylene glycols, polytetrahydro-furans, polydimethylsiloxanes, polyethylenes and polypropylenes. Polyethylene glycols are particularly preferred.

The linear molecules advantageously have, independently of one another, a weight-average molecular weight of greater than or equal to 350 g/mol, for example ranging from 350 to 2 000 000, preferably ranging from 1500 to 1 000 000, or preferably ranging from 2800 to 800 000, better still from 7000 to 700 000, for example ranging from 10 000 to 600 000 or from 10 000 to 500 000.

The linear molecules preferably carry reactive groups at each end. The fact of carrying the reactive groups makes it possible to facilitate the reaction with the molecular structures intended to prevent separation between the linear molecules and the cyclic molecules which they carry.

The reactive groups depend on the blocking molecular structures to be employed.

Mention may be made, as examples, of hydroxyl groups, amino groups, tosylate groups, polymerizable groups, activated ester groups, such as N-hydroxysuccinimide ester groups, carboxyl groups, thiol groups and the like.

### Cyclic Molecules

In the present invention, a “cyclic molecule” denotes a molecule comprising at least one cyclic structure. The cyclic molecule can comprise two or more cyclic structures or a double ring. The cyclic molecule can be a macrocycle, such as a cyclodextrin.

Examples of cyclic molecules in the present invention can comprise:
cyclodextrins, for example α-cyclodextrin, β-cyclodextrin, γ-cyclodextrin, dimethylcyclodextrin and glucosylcyclodextrin, and their derivatives,
crown ethers,
benzocrown ethers, dibenzocrown ethers and dicyclohexanocrown ethers,
and their derivatives.

The size of the internal cavity or cavities of the cyclic molecules can vary according to the linear molecule chosen. In any case, cyclic molecules are chosen which can be strung along the linear molecule. Thus, the cavity of the cyclic-molecule will preferably have a diameter greater than the diameter of the cross section of a minimum imaginary cylinder in which the linear molecule can be included.

When use is made of a cyclic molecule having a relatively large cavity and of a cylindrical linear molecule having a relatively small diameter, it is possible to include several linear molecules in the cavity of the cyclic molecule.

Preference is given, among the cyclic molecules which can be used, to cyclodextrins.

According to one embodiment, α-cyclodextrin is used as cyclic molecule and a polyethylene glycol is used as linear molecule.

The cyclic molecules preferably have groups capable of generating bonds which are not situated in their cavity. This makes it possible to subsequently bond the cyclic molecules to one another via a chemical or physical bond. The reactive groups of the cyclic molecules can comprise, for example, hydroxyl, amino, carboxyl or thiol groups. Furthermore, it is preferable to choose cyclic molecules having reactive groups which do not react with the blocking structures during the blocking reaction between the said blocking structures and the linear molecules.

The ratio of the number of cyclic molecules strung along a linear molecule to the maximum amount of cyclic molecules of the same nature which could be strung along this linear molecule ranges from 0.001 to 0.6, preferably from 0.01 to 0.5 and better still from 0.05 to 0.4. This ratio may be referred to as “inclusion amount”.

The maximum inclusion amount is standardized as being equal to 1. It corresponds to the amount at which a linear molecule makes it possible to include a maximum of cyclic molecules.

It is preferable for the linear molecule not to exhibit a dense stack of cyclic molecules. This dense stack state corresponding to the maximum inclusion amount equal to 1. The fact of creating a non-dense stack of cyclic molecules makes it possible to retain molecular segments which can be moved, with the result that the crosslinked polyrotaxane exhibits a high fracture strength, a high entropy elasticity, a superior expandability and/or a superior restoring property, and, if desired, a high absorbability or a high hygroscopicity.

According to another embodiment, the crosslinked polyrotaxane comprises cyclic molecules which each comprise at least two rings, in particular bicyclic molecules. In this embodiment, the linear molecule of the first polyrotaxane is threaded into the first ring of each bicyclic molecule and the linear molecule of the second polyrotaxane is threaded into the second ring of at least one bicyclic molecule. After mixing the linear molecules and the bicyclic molecules, each end of the linear molecules is blocked with a blocking group, so as to prevent the removal of the bicyclic molecules in their skewered state.

In this embodiment, it is not necessary to create bonds between the cyclic molecules via a crosslinking reaction since the covalent bond connecting the two rings of the bicyclic molecule in fact confers the crosslinking nature on the polyrotaxane.

The bicyclic molecule can comprise, in addition to the two main rings, one or more other nuclei.

According to one embodiment, the cyclic molecules can be cyclized after inclusion of the linear molecules.

More specifically, it is possible to use a precursor of the cyclic molecules having at least one open segment analogous to the letter “C”.

In this case, the “C” segments can be closed after the inclusion of the linear molecule or after the blocking of the linear molecule with a blocking group. For the molecules having a segment analogous to the letter “C”, see M. Asakawa et al., Angewandte Chemie International, 37(3), 333-337 (1998), and M. Asakawa et al., European Journal of Organic Chemistry, 5, 985-994 (1999), both being incorporated here by way of reference.

### Molecular Structures Situated at the Chain End of the Linear Molecules: Blocking Structures

The blocking structures have to keep the cyclic molecules strung along the linear molecule.

These blocking structures can prevent the cyclic molecules from separating from the linear molecule due to their high stearic volume.

The blocking structures situated at each end of each linear molecule can also prevent the cyclic molecules from decomplexing from the linear molecule by exhibiting specific ionic charges.

The expression “molecular structure” denotes here a molecule, a macromolecule or a solid support.

A macromolecule or a solid support can include several blocking sites.

A blocking structure of a macromolecule can be present in the main chain or in a side chain.

When a blocking structure is a macromolecule A, the macromolecule A can constitute a matrix, a portion of which comprises pseudopolyrotaxanes, or conversely the pseudopolyrotaxane can constitute a matrix, a portion of which comprises the macromolecule A.

The blocking molecular structures can be chosen from:
dinitrophenyl groups, such as the 2,4- and 3,5-dinitrophenyl groups:
cyclodextrins;
adamantane groups;
trityl groups;
fluoresceins;
pyrenes;
naphthalimides, and
their combinations.

According to one embodiment, when the linear molecule is a polyethylene glycol, the cyclic molecules can be chosen from α-cyclodextrin, dinitrophenyl groups, such as the 2,4- and 3,5-dintrophenyl groups, adamantane groups, trityl groups, fluoresceins, pyrenes and their combinations.

### Crosslinking: Cyclic or Bicyclic Molecules

According to one alternative form, the cyclic molecule is a bicyclic molecule. The crosslinked polyrotaxanes comprise at least one first polyrotaxane and one second polyrotaxane, the linear molecule of the first polyrotaxane being threaded into the first ring of a bicyclic molecule and the linear molecule of the second polyrotaxane being threaded into the second ring of at least one bicyclic molecule.

According to another alternative form, the crosslinked polyrotaxanes comprise at least one first polyrotaxane and one second polyrotaxane, at least one cyclic molecule of a first polyrotaxane and at least one cyclic molecule of a second polyrotaxane being bonded via at least one chemical or physical bond.

When the bond is a chemical bond, the chemical bond can be formed by a single bond or by a bond involving various atoms or molecules. The said bond can be obtained by reaction of the said two cyclic molecules with a crosslinking agent, a coupling agent or a photocrosslinking agent.

A cyclic molecule preferably has one or more reactive groups on the outside of the nucleus, as described above. In particular, it is preferable, after the formation of a blocked polyrotaxane molecule, for the cyclic molecules of different polyrotaxanes to be crosslinked with one another by means of a crosslinking agent. This reaction can be carried out under the action of the temperature or of a variation in pH. In this case, the conditions of the crosslinking reaction have to be conditions under which the blocking groups of the blocked polyrotaxane are not removed.

Use may be made, as crosslinking agents, of crosslinking agents well known in the prior art. Mention may be made, as examples, of cyanuryl chloride, trimesoyl chloride, terephthaloyl chloride, epichloro-hydrin, dibromobenzene, glutaraldehyde, phenylene diisocyanates, tolylene diisocyanates (for example, tolylene 2,4-diisocyanate), 1,1′-carbonyldiimidazole, divinyl sulphone, acid dichlorides (for example, sebacoyl dichloride), acids substituted by a trichloro group, and the like. Various types of coupling agents can also be incorporated, such as coupling agents of silane type (for example, various alkoxysilanes) and coupling agents based on titanium (for example, the various alkoxytitanium compounds). Mention may be made, as other examples, of various photocrosslinking agents which are employed for materials designed for soft contact lenses, for example photocrosslinking agents based on stilbazolium salts, such as formylstyryl-pyridinium salts (see K. Ichimura et al., Journal of Polymer Science, edition on the chemistry of polymers, 20, 1411-1432 (1982), incorporated here by way of reference), and other photocrosslinking agents, for example photocrosslinking agents by photodimerization, specifically cinnamic acid, anthracene, thymines and the like.

The crosslinking agents preferably have molecular weights of less than 2000 g/mol, preferably of less than 1000, better still of less than 600 and very particularly of less than 400.

In the case where α-cyclodextrin is used as cyclic molecule and where a crosslinking agent is used to crosslink it, mention may be made, as examples of crosslinking agent, of cyanuryl chloride, tolylene 2,4-diisocyanate, 1,1′-carbonyldiimidazole, trimesoyl chloride, terephthaloyl chloride, alkoxysilanes, such as tetramethoxysilane and tetraethoxysilane, and the like. In particular, it is preferable to use α-cyclodextrin as cyclic molecule and cyanuryl chloride as crosslinking agent.

### Preparation of a Polyrotaxane Crosslinked Via Chemical Bonds

The compounds according to the present invention can be prepared according to the teaching of Patent Application EP 1 283 218.

First of all, the cyclic molecules and the linear molecules are mixed in order to prepare the pseudopolyrotaxanes, in which the cyclic molecules are strung along the linear molecules. Secondly, the polyrotaxanes are prepared by blocking each end of the linear molecules with blocking groups, so as to prevent the removal of the cyclic molecules. Finally, two or more than two polyrotaxanes are crosslinked by bonding the cyclic molecules via chemical bonds, in order to obtain the crosslinked polyrotaxane.

According to one embodiment of the invention, α-cyclo-dextrin is used as cyclic molecule, a polyethylene glycol is used as linear molecule, a 2,4-dinitrophenyl group is used as blocking group and cyanuryl chloride is used as crosslinking agent.

First of all, each end of the polyethylene glycol is converted to an amino group, in order to be able subsequently to attach a blocking group to the end of the polyethylene glycol and to form the polyrotaxane. In an alternative form, use may be made of the diamine-terminated PEG/PPO copolymers sold by Huntsman under the Jeffamine reference.

Subsequently, the α-cyclodextrin and the aminated polyethylene glycol derivative are mixed in order to prepare the pseudopolyrotaxane. The duration of the mixing ranges from 1 to 48 hours and the mixing temperature ranges from 0 to 100° C., so that the inclusion amount of α-cyclodextrin with regard to the polyethylene glycol derivative ranges from 0.001 to 0.6.

Generally, a polyethylene glycol having an average molecular weight of 20 000 makes it possible to include at most 230 α-cyclodextrin molecules. The maximum inclusion amount, corresponding to 230 molecules, is equal to 1.

According to one embodiment, 60 to 65 (63) α-cyclo-dextrin molecules are on average strung over one polyethylene glycol molecule, which corresponds to a degree of inclusion ranging from 0.26 to 0.29 (0.28) with respect to the maximum inclusion amount. The α-cyclodextrin inclusion amount can be determined by NMR, light absorption or elemental analysis.

The pseudopolyrotaxane obtained is reacted with 2,4-dinitrofluorobenzene dissolved in DMF, which makes it possible to obtain the polyrotaxane.

The polyrotaxane is subsequently dissolved in an aqueous sodium hydroxide solution and then cyanuryl chloride is added in order to crosslink the α-cyclodextrins.

The cosmetic composition according to the present invention can comprise one or more crosslinked polyrotaxanes in a content ranging from 0.1 to 80% by weight, preferably from 1 to 30% by weight and more preferably from 3 to 25% by weight, with respect to the total weight of the composition.

The cosmetic composition in accordance with the present invention can comprise at least one oil. It can additionally comprise another fatty substance chosen from waxes and pasty fatty substances.

The term “oil” is understood to mean any fatty substance in the liquid form at ambient temperature (20-25° C.) and at atmospheric pressure. The liquid fatty phase can also comprise, in addition to oils, other compounds dissolved in the oils, such as gelling and/or structuring agents.

The oil or oils can be present in a proportion of 0.1 to 99.9% by weight, in particular of at least 1 to 90% by weight, more particularly of 5 to 70% by weight, especially of 10 to 60% by weight, indeed even of 20 to 50% by weight, with respect to the total weight of the cosmetic composition according to the invention.

The oil or oils can be volatile or non-volatile and hydrocarbon or silicone oils.

Within the meaning of the present invention, the term “volatile oil” is understood to mean an oil (or non-aqueous medium) capable of evaporating on contact with the skin in less than one hour at ambient temperature and at atmospheric pressure. The volatile oil is a volatile cosmetic oil which is liquid at ambient temperature and which has in particular a non-zero vapour pressure at ambient temperature and atmospheric pressure, especially a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

Within the meaning of the present invention, the term “non-volatile oil” is understood to mean an oil having a vapour pressure of less than 0.13 Pa.

The volatile or non-volatile oils can be hydrocarbon oils, in particular of animal or vegetable origin, synthetic oils, silicone oils, fluorinated oils or their mixtures.

Within the meaning of the present invention, the term “silicone oil” is understood to mean an oil comprising at least one silicon atom and in particular at least one Si—O group.

The term “hydrocarbon oil” is understood to mean an oil comprising mainly hydrogen and carbon atoms and optionally oxygen, nitrogen, sulphur and/or phosphorus atoms.

The volatile hydrocarbon oils can be chosen from hydrocarbon oils having from 8 to 16 carbon atoms and in particular branched C₈-C₁₆ alkanes (also referred to as isoparaffins), such as isododecane (also referred to as 2,2,4,4,6-pentamethylheptane), isodecane, isohexa-decane and, for example, the oils sold under the Isopar or Permethyl trade names.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 10 silicon atoms, especially from 2 to 7 silicon atoms, these silicones optionally comprising alkyl or alkoxy groups having from 1 to 10 carbon atoms. Mention may in particular be made, as volatile silicone oil which can be used in the invention, of dimethicones with viscosities of 5 and 6 cSt, octamethylcyclotetrasiloxane, decamethylcyclo-pentasiloxane, dodecamethylcyclohexasiloxane, hepta-methylhexyltrisiloxane, heptamethyloctyltrisiloxane, hexamethyldisiloxane, octamethyltrisiloxane, deca-methyltetrasiloxane, dodecamethylpentasiloxane and their mixtures.

Use may also be made of volatile fluorinated oils, such as nonafluoromethoxybutane or perfluoromethylcyclo-pentane, and their mixtures.

The non-volatile oils can be chosen in particular from non-volatile hydrocarbon oils, if appropriate fluorinated, and/or non-volatile silicone oils.

Mention may in particular be made, as non-volatile hydrocarbon oil, of:
hydrocarbon oils of animal origin,
hydrocarbon oils of vegetable origin, such as phytosteryl esters, for example phytosteryl oleate, phytosteryl isostearate and lauroyl/octyldodecyl/phytosteryl glutamate (Ajinomoto, Eldew PS203), triglycerides composed of esters of fatty acids and of glycerol, the fatty acids of which can have varied chain lengths from C₄ to C₂₄, it being possible for these chains to be linear or branched and saturated or unsaturated; these oils are in particular heptanoic or octanoic triglycerides; wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam®, squalane and their mixtures, in particular hydrogenated polyisobutene,
synthetic esters, such as oils of formula R₁COOR₂ in which R₁ represents the residue of a linear or branched acid comprising from 1 to 40 carbon atoms, and R₂ represents a hydrocarbon chain, in particular a branched hydrocarbon chain, comprising from 1 to 40 carbon atoms, provided that R₁+R₂≧10.

These esters can in particular be chosen from cetearyl octanoate, esters of isopropyl alcohol, such as isopropyl myristate or isopropyl palmitate, ethyl palmitate, 2-ethylhexyl palmitate, isopropyl stearate or isostearate, isostearyl isostearate, octyl stearate, hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and their mixtures, C₁₂ to C₁₅ alkyl benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, or hydroxylated esters, such as isostearyl lactate or diisostearyl malate;
esters of polyols and esters of pentaerythritol, such as dipentaerythritol tetrahydroxystearate/tetra-isostearate,
esters of dimer diols and dimer diacids, such as Lusplan DD-DA5® and Lusplan DD-DA7®, sold by Nippon Fine Chemical and disclosed in Application FR0302809 filed on 6 Mar. 2003, the content of which is incorporated in the present application by way of reference,
fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpenta-decanol,
higher fatty acids, such as oleic acid, linoleic acid, linolenic acid and their mixtures, and
dialkyl carbonates, it being possible for the 2 alkyl chains to be identical or different, such as dicaprylyl carbonate, sold under the name Cetiol CC® by Cognis.

The non-volatile silicone oils which can be used in the composition according to the invention can be non-volatile polydimethylsiloxanes (PDMSs), polydimethyl-siloxanes comprising pendent alkyl or alkoxy groups and/or alkyl or alkoxy groups at the ends of the silicone chain, which groups each have from 2 to 24 carbon atoms, phenylated silicones, such as phenyl trimethicones, phenyl dimethicones, phenyl(trimethyl-siloxy)diphenylsiloxanes, diphenyl dimethicones, diphenyl(methyldiphenyl)trisiloxanes and (2-phenyl-ethyl)trimethylsiloxysilicates, dimethicones or phenyl trimethicones with a viscosity of less than or equal to 100 cSt, and their mixtures.

The non-volatile oils can be present in the compositions according to the invention in a content ranging from 20% to 99.9% by weight, in particular from 30% to 80% by weight and especially from 40% to 80% by weight, with respect to the total weight of the composition.

The composition can also comprise a pasty fatty substance and/or a wax.

The term “pasty fatty substance” is understood to mean a lipophilic compound comprising, at a temperature of 23° C., a liquid fraction and a solid fraction. The term “pasty fatty substance” is also understood to mean poly(vinyl laurate).

The term “wax”, within the meaning of the present invention, is understood to denote a lipophilic compound which is solid at ambient temperature (25° C.), which exhibits a reversible solid/liquid change in state and which has a melting point of greater than or equal to 30° C. which can reach up to 120° C.

The melting point of the wax can be measured using a differential scanning calorimeter (DSC), for example the calorimeter sold under the name DSC 30 by Mettler.

The waxes can be hydrocarbon, fluorinated and/or silicone waxes and be of vegetable, mineral, animal and/or synthetic origin. In particular, the waxes exhibit a melting point of greater than 25° C. and better still of greater than 45° C.

Mention may be made, as waxes which can be used in the first composition, of linear hydrocarbon waxes. Their melting point is advantageously greater than 35° C., for example greater than 55° C. and preferably greater than 80° C.

The linear hydrocarbon waxes are advantageously chosen from substituted linear alkanes, unsubstituted linear alkanes, unsubstituted linear alkenes or substituted linear alkenes, an unsubstituted compound being composed solely of carbon and hydrogen. The substituents mentioned above not comprising carbon atoms.

The linear hydrocarbon waxes include polymers and copolymers of ethylene with a molecular weight of between 400 and 800, for example the Polywax 500 or Polywax 400 sold by New Phase Technologies.

The linear hydrocarbon waxes include linear paraffin waxes, such as the paraffin waxes S&P 206, S&P 173 and S&P 434 from Strahl & Pitsch.

The linear hydrocarbon waxes include long-chain linear alcohols, such as the products comprising a mixture of polyethylene and of alcohols comprising 20 to 50 carbon atoms, in particular the Performacol 425 or Performacol 550 (mixture in proportions 20/80) sold by New Phase Technologies.

Examples of silicone waxes are, for example
the C₂₀₋₂₄ alkyl methicone, C₂₄₋₂₈ alkyl dimethicone, C₂₀₋₂₄ alkyl dimethicone and C₂₄₋₂₈ alkyl dimethicone sold by Archimica Fine Chemicals under the reference SilCare 41M40, SilCare 41M50, SilCare 41M70 and SilCare 41M80,
the stearyl dimethicones with the reference SilCare 41M65 sold by Archimica or with the reference DC-2503 sold by Dow Corning,
the stearoxytrimethylsilanes sold under the reference SilCare 1M71 or DC-580,
the products Abil Wax 9810, 9800 or 2440 from Wacker Chimie GmbH,

the C₃₀₋₄₅ alkyl methicone sold by Dow Corning under the reference AMS-C30 Wax and the C₃₀₋₄₅ alkyl dimethicones sold under the reference SF1642 or SF1632 by General Electric.

The nature and the amount of these fatty substances depend on the mechanical properties and textures desired.

The composition according to the invention can be used for making up and/or caring for keratinous substances other than the hair, such as the skin (of the face, of the body, the scalp, the lips), the eyelashes and the eyebrows.

The cosmetic composition according to the invention can be provided in the form of a product for making up and/or caring for keratinous substances and in particular the skin, in particular in the form of a foundation, and the lips, in particular a lipstick or a lip balm.

The composition has a particular application as composition for caring for the body or face or composition for making up the body or face, such as foundation, lipstick, lipcare product, nail varnish, nailcare product, mascara or eyeliner.

The composition according to the invention can comprise a colouring material.

The colouring material can be any inorganic and/or organic compound exhibiting an absorption between 350 and 700 nm or capable of generating an optical effect, such as the reflection of incident light or interferences, for example.

The colouring materials of use in the present invention are chosen from all the organic and/or inorganic pigments known in the art, in particular those which are described in the Kirk-Othmer Encyclopaedia of Chemical Technology and in Ullmann's Encyclopaedia of Industrial Chemistry.

For a composition in the paste or cast form, such as lipsticks or make-up products, use is generally made of 0.5 to 50% of colouring material, preferably of 2 to 40% and better still of 5 to 30%, with respect to the total weight of the composition.

Mention may be made, as examples of inorganic colouring materials, of titanium dioxide, which is or is not surface treated, zinc oxide, zirconium or cerium oxides, iron or chromium oxides, manganese violet, ultramarine blue, chromium hydrate and ferric blue. For example, the following inorganic pigments can be used: Ta₂O₅, Ti₃O₅, Ti₂O₃, TiO, ZrO₂ as a mixture with TiO₂, ZrO₂, Bn₂O₅, CeO₂ or ZnS.

Mention may be made, as examples of organic colouring materials, of nitroso, nitro, azo, xanthene, quinoline, anthaquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

In particular, the colouring materials can be chosen from carmine, carbon black, aniline black, azo yellow, quinacridone, phthalocyanine blue, sorghum red, the blue pigments classified in the Colour Index under the references CI 42090, 69800, 69825, 73000, 74100 and 74160, the yellow pigments classified in the Colour Index under the references CI 11680, 11710, 15985, 19140, 20040, 21100, 21108, 47000 and 47005, the green pigments classified in the Colour Index under the references CI-61565, 61570 and 74260, the orange pigments classified in the Colour Index under the references CI 11725, 15510, 45370 and 71105, the red pigments classified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives, as disclosed in Patent FR 2 679 771.

The pigments in accordance with the invention can also be in the form of composite pigments, as disclosed in Patent EP 1 184 426. These composite pigments can be composed in particular of particles comprising an inorganic core, at least one binder, which provides for the attachment of the organic pigments to the core, and at least one organic pigment at least partially covering the core.

The colouring materials can be chosen from dyes, lakes or pigments.

The dyes are, for example, fat-soluble dyes, although water-soluble dyes may be used. The fat-soluble dyes are, for example Sudan Red, D & C Red 17, D & C Green 6, β-carotene, soybean oil, Sudan Brown, D & C Yellow 11, D & C Violet 2, D & C Orange 5, quinoline yellow or annatto. They can represent from 0 to 20% of the weight of the composition and better still from 0.1 to 6%. The water-soluble dyes are in particular beetroot juice or methylene blue and can represent from 0.1 to 6% by weight of the composition (if present).

The term “lake” is understood to mean dyes adsorbed on insoluble particles, the combination thus obtained remaining insoluble when used. The inorganic substrates on which the dyes are adsorbed are, for example, alumina, silica, calcium sodium borosilicate, calcium aluminium borosilicate and aluminium. Mention may be made, among organic dyes, of cochineal carmine.

Mention may be made, as examples of lakes, of the products known under the following names: D & C Red 21 (CI 45 380). D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053) or D & C Blue 1 (CI 42 090).

The term “pigments” should be understood as meaning white or coloured and inorganic or organic particles intended to colour and/or opacify the composition. The pigments in accordance with the invention can, for example, be chosen from white or coloured pigments or from pigments possessing special effects, such as pearlescent agents, reflective pigments or interference pigments.

Mention may be made, as pigments which can be used in the invention, of titanium, zirconium or cerium oxides as well as zinc, iron or chromium oxides and ferric blue. Mention may be made, among the organic pigments which can be used in the invention, of carbon black and barium, strontium, calcium (D & C Red No. 7) and aluminium lakes.

The pearlescent agents can be present in the composition in a proportion of 0.001 to 20% of the total weight of the composition, preferably at a level of the order of 1 to 15%. Mention may be made, among the pearlescent agents which can be used in the invention, of mica covered with titanium oxide, with iron oxide, with natural pigment or with bismuth oxychloride, such as coloured titanium oxide-coated mica.

The pigments can be present in the composition in a proportion of 0.05 to 30% of the weight of the final composition and preferably in a proportion of 2 to 20%.

The variety of the pigments which can be used in the present invention makes it possible to obtain a rich pallet of colours and also specific optical effects, such as metallic or interference effects.

The term “pigments possessing special effects” is understood to mean pigments which generally create a coloured appearance (characterized by a certain hue, a certain saturation and a certain lightness) which is non-uniform and which changes according to the conditions of observation (light, temperature, angles of observation, and the like). They consequently contrast with white or coloured pigments, which provide a conventional opaque, semitransparent or transparent uniform colouring.

Mention may be made, as examples of pigments possessing special effects, of white pearlescent pigments, such as mica covered with titanium dioxide or with bismuth oxychloride, coloured pearlescent pigments, such as mica covered with titanium dioxide and with iron oxides, mica covered with titanium dioxide and in particular with ferric blue or with chromium oxide or mica covered with titanium dioxide and with an organic pigment as defined above, and pearlescent pigments based on bismuth oxychloride. Mention may be made, as pearlescent pigments, of the following pearlescent agents, Cellini, sold by Engelhard (mica-TiO₂-lake), Prestige, sold by Eckart (mica-TiO₂), or Colorona, sold by Merck (mica-TiO₂—Fe₂O₃).

Mention may also be made of pigments possessing an interference effect which are not attached to a substrate, such as liquid crystals (Helicones HC from Wacker) or holographic interference flakes (Geometric Pigments or Spectra f/x from Spectratek). Pigments possessing special effects also comprise fluorescent pigments, whether it be substances which are fluorescent in daylight or which produce ultraviolet fluorescence, phosphorescent pigments, photochromic pigments and thermochromic pigments.

The composition advantageously comprises goniochromatic pigments, for example multilayer interference pigments, and/or reflective pigments. These two types of pigments are disclosed in Application FR 0 209 246, the content of which is incorporated by reference in the present application.

The composition can comprise reflective pigments which may or may not be goniochromatic pigments and which may or may not be interference pigments.

Their size is compatible with the demonstration of a specula reflection of visible light (400-700 nm) of sufficient intensity, taking into account the mean gloss of the composition, to create a highlight point.

This size is capable of varying according to the chemical nature of the particles, their shape and their capacity for specula reflection of visible light.

The reflective particles will preferably exhibit a dimension of at least 10 μm, for example of between approximately 20 μm and approximately 50 μm.

The term “dimension” denotes the dimension given by the statistical particle size distribution to half the population, referred to as D50. The size of the reflective particles can depend on their surface condition. The more reflective the latter, the smaller may a priori be the dimension, and vice versa.

Reflective particles usable in the invention, possessing a metallic or white glint, can, for example, reflect the light in all the components of the visible region without significantly absorbing one or more wavelengths. The spectral reflectance of these reflective particles can, for example be greater than 70% within the 400-700 nm range and better still at least 80%, indeed even 90% or also 95%.

The reflective particles, whatever their shape, may or may not exhibit a multilayer structure and, in the case of a multilayer structure, may exhibit, for example, at least one layer of uniform thickness, in particular of a reflective material, which coats a substrate.

The substrate can be chosen from glasses, ceramics, graphite, metal oxides, aluminas, silicas, silicates, in particular aluminosilicates and borosilicates, and synthetic mica, this list not being limiting.

The reflective material can comprise a layer of metal or of a metal compound.

The layer of metal or of metal compound may or may not completely coat the substrate and the layer of metal may be at least partially covered with a layer of another material, for example a transparent material.

It may be preferable for the layer of metal or of metal compound to completely coat the substrate, directly or indirectly, that is to say with insertion of at least one intermediate metal or non-metal layer.

The metal can be chosen, for example, from Ag, Au, Cu, Al, Ni, Sn, Mg, Cr, Mo, Ti, Pt, Va, Rb, W, Zn, Ge, Te, Se and their alloys. Ag, Au, Al, Zn, Ni, Mo, Cr, Cu and their alloys (for example, bronzes and brasses) are preferred metals.

In the case in particular of particles possessing a substrate coated with silver or with gold, the metal layer can be present at a content representing, for example, from 0.1 to 50% of the total weight of the particles, indeed even between 1 and 20%.

Particles of glass covered with a metal layer are disclosed in particular in the documents JP-A-09188830, JP-A-10158450, JP-A-10158541, JP-A-07258460 and JP-A-05017710.

Particles possessing a glass substrate coated with silver, in the form of platelets, are sold under the name Microglass Metashine REFSX 2025 PS by Toyal.

Particles possessing a glass substrate coated with nickel/chromium/molybdenum alloy are sold under the name Crystal Star GF 550 or GF 2525 by this same company.

The reflective particles, whatever their shape, can also be chosen from particles possessing a synthetic substrate at least partially coated with at least one layer of at least one metal compound, in particular a metal oxide, for example chosen from titanium oxides, in particular TiO₂, iron oxides; in particular Fe₂O₃, tin oxides, chromium oxides, barium sulphate and the following compounds: MgF₂, CrF₃, ZnS, ZnSe, SiO₂, Al₂O₃, MgO, Y₂O₃, SeO3, SiO, HfO₂, ZrO₂, CeO₂, Nb2O₅, Ta₂O₅, MoS₂ and their mixtures or alloys.

Mention may be made, as examples of such particles, for example, of particles comprising a substrate of synthetic mica coated with titanium dioxide or particles of glass coated either with brown iron oxide or with titanium oxide, with tin oxide or with one of their mixtures, such as those sold under the Reflecks® brand by Engelhard.

Pigments of the Metashine 1080R range, sold by Nippon Sheet Glass Co. Ltd., are also suitable. These pigments, more particularly disclosed in Patent Application JP 2001-11340, are flakes of C-Glass glass comprising 65 to 72% of SiO₂ which are covered with a layer of titanium oxide of rutile (TiO₂) type. These glass flakes have a mean thickness of 1 micron and a mean size of 80 microns, i.e. a mean size/mean thickness ratio of 80. They exhibit blue, green, yellow or silver-coloured glints, depending on the thickness of the TiO₂ layer.

Mention may also be made of particles with a dimension of between 80 and 100 μm comprising a substrate of synthetic mica (fluorophlogopite) coated with titanium dioxide representing 12% of the total weight of the particle, these particles being sold under the name Prominence by Nihon Koken.

The reflective particles can also be chosen from particles formed of a stack of at least two layers possessing different refractive indices. These layers can be polymeric or metallic in nature and can in particular include at least one polymer layer. Such particles are disclosed in particular in WO 99/36477, U.S. Pat. No. 6,299,979 and U.S. Pat. No. 6,387,498. Mention may be made, by way of illustration of the materials which can constitute the various layers of the multilayer structure, of, this list not being limiting: polyethylene naphthalate (PEN) and its isomers, poly(alkylene terephthalate)s and polyimides.

Reflective particles comprising a stack of at least two layers of polymers are sold by 3M under the name Mirror Glitter. These particles comprise layers of 2,6-PEN and of poly(methyl methacrylate) in a ratio by weight of 80/20. Such particles are disclosed in U.S. Pat. No. 5,825,643.

The composition can comprise one or more goniochromatic pigments.

The goniochromatic colouring agent can be chosen, for example, from multilayer interference structures and liquid crystal colouring agents.

In the case of a multilayer structure, the latter can comprise, for example, at least two layers, each layer, independently or not independently of the other layer(s), being produced, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MOS₂, cryolite, alloys, polymers and their combinations.

The multilayer structure may or may not exhibit, with respect to a central layer, a symmetry with regard to the chemical nature of the stacked layers.

Examples of symmetrical multilayer interference structures which can be used are, for example, the following structures: Al/SiO₂/Al/SiO₂/Al, pigments having this structure being sold by DuPont de Nemours; Cr/MgF₂/Al/MgF₂/Cr, pigments having this structure being sold under the name Chromaflair by Flex; MOS₂/SiO₂/Al/SiO₂/MOS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by BASF; MOS₂/SiO₂/mica/SiO₂/MOS₂; Fe₂O₃/SiO₂/mica/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by Merck (Darmstadt). By way of examples, these pigments can be pigments with a silica/titanium oxide/tin oxide structure sold under the name Xirona Magic by Merck, pigments with a silica/brown iron oxide structure sold under the name Xirona Indian Summer by Merck and pigments with a silica/titanium oxide/mica/tin oxide structure sold under the name Xirona Caribbean Blue by Merck. Mention may also be made of the Infinite Colors pigments from Shiseido. Different effects are obtained according to the thickness and the nature of the various layers. Thus, with the structure Fe₂O₃/SiO₂/Al/SiO₂/Fe₂O₃, the colour changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Use may also be made of goniochromatic colouring agents possessing a multilayer structure comprising an alternation of polymer layers, for example of the polyethylene naphthalate and polyethylene terephthalate type. Such agents are disclosed in particular in WO-A-96/19347 and WO-A-99/36478.

Mention may be made, as examples of pigments possessing a polymeric multilayered structure, or those sold by 3M under the name Color Glitter.

The liquid crystal colouring agents comprise, for example, silicones or cellulose ethers onto which mesomorphic groups are grafted.

Use may be made, as liquid crystal goniochromatic particles, for example, of those sold by Chemx and of those sold under the name Helicone® HC by Wacker.

The composition can additionally comprise disperse goniochromatic fibres. Such fibres can, for example, exhibit a size of between 200 μm and 700 μm, for example of approximately 300 μm.

Use may in particular be made of interference fibres possessing a multilayer structure. Fibres possessing a multilayer structure of polymers are disclosed in particular in the documents EP-A-921 217, EP-A-686 858 and U.S. Pat. No. 5,472,798. The multilayer structure can comprise at least two layers, each layer, independently or not independently of the other layer(s), being made of at least one synthetic polymer. The polymers present in the fibres can have a refractive index ranging from 1.30 to 1.82 and better still ranging from 1.35 to 1.75. The preferred polymers for forming the fibres are polyesters, such as polyethylene terephthalate, polyethylene naphthalate or polycarbonate, acrylic polymers, such as poly(methyl methacrylate), or polyamides.

Goniochromatic fibres possessing a polyethylene terephthalate/nylon-6 two-layer structure are sold by Teijin under the name Morphotex.

The composition according to the invention can comprise fillers.

The term “fillers” should be understood as meaning colourless or white, inorganic or synthetic and lamellar or non-lamellar particles. These fillers and pearlescent agents are used in particular to modify the texture of the composition and are included in particular among the structuring agents capable of resulting in a solid form.

The fillers can be present in a proportion of 0 to 60% of the total weight of the composition, preferably 0.5 to 20%. Mention may in particular be made of talc, mica, kaolin, powders formed of nylon (in particular orgasol) and of polyethylene, Teflon, starch, boron nitride, microspheres formed of copolymers, such as Expancel (Nobel Industrie) or Polytrap (Dow Corning), and silicone resin microbeads (Tospearl from Toshiba, for example).

A further subject-matter of the invention is a cosmetic process for making up and/or caring for keratinous substances other than the hair, such as the skin, the lips or the eyelashes, comprising the application, to the keratinous substances, of the cosmetic composition as defined above.

Another subject-matter of the invention is the use of at least one crosslinked polyrotaxane in an anhydrous cosmetic composition for remodelling the face, the body or the lips, increasing the volume of the lips, concealing imperfections or defects in the appearance of keratinous substances, rendering the complexion uniform or increasing the volume of the eyelashes.

The invention is illustrated in more detail in the following examples. The percentages are given by weight.

### Preparation of the Crosslinked Polyrotaxane

0.9 g of polyethylene glycol bisamine (abbreviated to PEG-BA), sold by Fluka, and 3.6 g of α-cyclodextrin were dissolved in 30 ml of water at 80° C. and the mixture was maintained at 5° C. overnight in order to obtain the white paste of the inclusion complex.

The paste was dried, an excess of 2,4-dinitro-fluorobenzene (2.4 ml) was added at the same time as 10 ml of dimethylformamide, and then the mixture was stirred in a nitrogen atmosphere at ambient temperature overnight. The reaction mixture was dissolved in 50 ml of DMSO and precipitated twice from a 0.1% aqueous sodium chloride solution (800 ml) to give a yellow product. The product was collected, washed with water and methanol (three times, respectively) and dried to produce the polyrotaxane (1.25 g).

100 mg of polyrotaxane were dissolved in 0.5 ml of 1N NaOH at 5° C. in a reactor. 35 mg of 2,4,6-trichloro-1,3,5-triazine, dissolved in 0.5 ml of 1N NaOH, were added to the reactor. The reaction mixture was reacted at ambient temperature for 3 hours to produce the crosslinked polyrotaxane.

In the examples which follow, the percentages are by weight.

### EXAMPLE 1

### Glossy Base for the Lips

| | |
|---|---|
| Polybutene | 89.5% |
| Crosslinked polyrotaxane of the above | 10% |
| example | |
| Fragrance | q.s. for 100 |

### EXAMPLE 2

### Gloss for the Lips

| | |
|---|---|
| Polybutene | 86.5% |
| Crosslinked polyrotaxane of the above | 10% |
| example | |
| Pigments | 3% |
| Fragrance | q.s. for 100 |

### EXAMPLE 3

### Glossy Base for the Lips

| | |
|---|---|
| Polyisobutene | 74.5% |
| Octyldodecanol | 15% |
| Crosslinked polyrotaxane of the above | 10% |
| example | |
| Fragrance | q.s. for 100 |

### EXAMPLE 4

### Gloss for the Lips

| | |
|---|---|
| Polyisobutene | 71.5% |
| Octyldodecanol | 15% |
| Crosslinked polyrotaxane of the above | 10% |
| example | |
| Pigments | 3% |
| Fragrance | q.s. for 100 |

### EXAMPLE 5

### Mascara

| | |
|---|---|
| Beeswax | 10% |
| Carnauba wax | 7% |
| Allyl stearate/VA copolymer | 2.2% |
| (Mexomer PQ, manufactured by Chimex) | |
| Distearylammonium-modified hectorite | 5.32% |
| (Bentone 38VCG from Elementis) | |
| Propylene carbonate | 1.74% |
| Crosslinked polyrotaxane of the above | 7% |
| example | |
| Black iron oxide | 5% |
| Preservative | 0.2% |
| Isododecane | q.s. for 100 |

### EXAMPLE 6

### Stick Anhydrous Foundation

| | |
|---|---|
| Polyethylene wax (Mw: 500) | 4.5% |
| Polyethylene wax (Mw: 400) | 9.5% |
| Cyclomethicone D6 | 23% |
| Crosslinked polyrotaxane of the above | 5% |
| example | |
| Phenyl trimethicone (DC 556) | 22% |
| Iron oxide | 3.1% |
| Titanium dioxide | 10.9% |
| PMMA | 6% |
| Cyclomethicone D5 | 16% |

1-35. (canceled)

36. An anhydrous cosmetic composition comprising at least one crosslinked polyrotaxane and at least one oil.

37. The anhydrous cosmetic composition according to claim 36, wherein the at least one crosslinked polyrotaxane comprises at least one first polyrotaxane and at least one second polyrotaxane, each polyrotaxane comprising at least one independently chosen linear molecule and at least two independently chosen cyclic molecules, wherein at least one cyclic molecule of the at least one first polyrotaxane and at least one cyclic molecule of the at least one second polyrotaxane are bonded via at least one bond chosen from chemical bonds and physical bonds.

38. The anhydrous cosmetic composition according to claim 36, wherein the at least one crosslinked polyrotaxane comprises at least one first polyrotaxane and at least one second polyrotaxane, each polyrotaxane comprising at least one linear molecule and at least two cyclic molecules, wherein the at least one linear molecule of the at least one first polyrotaxane being threaded into a first ring of at least one bicyclic molecule and the at least one linear molecule of the at least one second polyrotaxane is threaded into a second ring of the at least one bicyclic molecule.

39. The anhydrous cosmetic composition according to claim 37, wherein the at least one linear molecule of at least one polyrotaxane chosen from the at least one first polyrotaxane and the at least one second polyrotaxane is chosen, independently of one another, from polymers.

40. The anhydrous cosmetic composition according to claim 39, wherein the at least one linear molecule of the at least one first polyrotaxane and the at least one linear molecule of the at least one second polyrotaxane are chosen, independently of one another, from:
hydrophilic polymers;
polyacrylamides;
polyalkylene glycols;
hydrophobic polymers; and
derivatives and copolymers thereof.

41. The anhydrous cosmetic composition according to claim 40, wherein the hydrophilic polymers are chosen from poly(vinyl alcohol)s, polyvinylpyrrolidones, poly((meth)acrylic acid)s, polymers derived from cellulose, polytetrahydrofurans, poly(vinyl acetal)s, poly(vinyl methyl ether)s, polyamines, polyethyleneimine, caseins, gelatins, and starchs.

42. The anhydrous cosmetic composition according to claim 41, wherein the polymers derived from cellulose are chosen from carboxymethylcellulose, hydroxyethylcellulose, and hydroxypropyl cellulose.

43. The anhydrous cosmetic composition according to claim 40, wherein the polyalkylene glycols are chosen from polyethylene glycols and polypropylene glycols.

44. The anhydrous cosmetic composition according to claim 40, wherein the hydrophobic polymers are chosen from polyolefins, copolymers of olefins, polyesters, polydimethylsiloxanes, poly(vinyl chloride), polystyrene, acrylonitrile/styrene copolymers, polymers of (meth)acrylic esters, copolymers of (meth)acrylic esters, polycarbonates, polyurethanes, vinyl chloride/vinyl acetate copolymers and poly(vinyl butyral).

45. The anhydrous cosmetic composition according to claim 44, wherein the polyolefins are chosen from polyethylenes, polypropylenes, polyisoprenes, polyisobutylenes, and polybutadienes.

46. The anhydrous cosmetic composition according to claim 44, wherein the copolymers of olefins are chosen from ethylene/butylene copolymers.

47. The anhydrous cosmetic composition according to claim 44, wherein the polymers and copolymers of (meth)acrylic esters are chosen from poly (methyl methacrylate) polymers and acrylonitrile/methyl acrylate copolymers.

48. The anhydrous cosmetic composition according to claim 40, wherein the at least one linear molecule of the at least one first polyrotaxane and the at least one linear molecule of the at least one second polyrotaxane are chosen, independently of one another, from polyethylene glycols, polyisoprenes, polyisobutylenes, polybutadienes, polypropylene glycols, polytetrahydrofurans, polydimethylsiloxanes, polyethylenes, and polypropylenes.

49. The anhydrous cosmetic composition according to claim 48, wherein the at least one linear molecule of the at least one first polyrotaxane and the linear molecule of the at least one second polyrotaxane are chosen, independently of one another, from polyethylene glycols and polypropylene glycols.

50. The anhydrous cosmetic composition according to claim 37, wherein at least one linear molecule chosen from at least one linear molecule of the at least one first polyrotaxane and the at least one linear molecule of the at least one second polyrotaxane have, independently of one another, a weight-average molecular weight of greater than or equal to 350 g/mol.

51. The anhydrous cosmetic composition according to claim 37, wherein at least one linear molecule chosen from at least one linear molecule of the at least one first polyrotaxane and the at least one linear molecule of the at least one second polyrotaxane have, independently of one another, a weight-average molecular weight of ranging from 350 g/mol to 2,000,000 g/mol.

52. The anhydrous cosmetic composition according to claim 51, wherein at least one linear molecule chosen from at least one linear molecule of the at least one first polyrotaxane and the at least one linear molecule of the at least one second polyrotaxane have, independently of one another, a weight-average molecular weight ranging from 1,500 g/mol to 1,000,000 g/mol.

53. The anhydrous cosmetic composition according to claim 52, wherein at least one linear molecule chosen from at least one linear molecule of the at least one first polyrotaxane and the at least one linear molecule of the at least one second polyrotaxane have, independently of one another, a weight-average molecular weight 2,800 g/mol to 800,000 g/mol.

54. The anhydrous cosmetic composition according to claim 53, wherein at least one linear molecule chosen from at least one linear molecule of the at least one first polyrotaxane and the at least one linear molecule of the at least one second polyrotaxane have, independently of one another, a weight-average molecular weight ranging from 7,000 g/mol to 700,000 g/mol.

55. The anhydrous cosmetic composition according to claim 54, wherein at least one linear molecule chosen from at least one linear molecule of the at least one first polyrotaxane and the at least one linear molecule of the at least one second polyrotaxane have, independently of one another, a weight-average molecular weight ranging from 10,000 g/mol to 600,000 g/mol.

56. The anhydrous cosmetic composition according to claim 55, wherein at least one linear molecule chosen from at least one linear molecule of the at least one first polyrotaxane and the at least one linear molecule of the at least one second polyrotaxane have, independently of one another, a weight-average molecular weight ranging from 10,000 g/mol to 500,000 g/mol.

57. The anhydrous cosmetic composition according to claim 37, wherein at least one linear molecule chosen from the at least one linear molecule of the at least one first polyrotaxane and the at least one linear molecule of the at least one second polyrotaxane carry, independently of one another, reactive groups chosen from hydroxyl groups, amino groups, tosylate groups, polymerizable groups, activated ester groups, carboxyl groups, and thiol groups.

58. The anhydrous cosmetic composition according to claim 37, wherein the at least two cyclic molecules of the at least one first polyrotaxane and of the at least one second polyrotaxane are chosen from:
cyclodextrins;
crown ethers;
benzocrown ethers;
dibenzocrown ethers;
dicyclohexanocrown ethers; and
their derivatives.

59. The anhydrous cosmetic composition according to claim 58, wherein the cyclodextrins are chosen from α-cyclodextrins, β-cyclodextrins, γ-cyclodextrins, dimethylcyclodextrins, and glucosylcyclodextrins.

60. The anhydrous cosmetic composition according to claim 59, wherein the cyclodextrins are chosen from α-cyclodextrins.

61. The anhydrous cosmetic composition according to claim 37, wherein the ratio of the number of cyclic molecules strung along the linear molecule of each polyrotaxane to the maximum amount of cyclic molecules of the same nature which could be strung along the linear molecule ranges from 0.001 to 0.6.

62. The anhydrous cosmetic composition according to claim 61, wherein the ratio of the number of cyclic molecules strung along the linear molecule of each polyrotaxane to the maximum amount of cyclic molecules of the same nature which could be strung along the linear molecule ranges from 0.01 to 0.5.

63. The anhydrous cosmetic composition according to claim 62, wherein the ratio of the number of cyclic molecules strung along the linear molecule of each polyrotaxane to the maximum amount of cyclic molecules of the same nature which could be strung along the linear molecule ranges from 0.05 to 0.4.

64. The anhydrous cosmetic composition according to claim 37, wherein the at least one linear molecule of the first polyrotaxane and the at least one linear molecule of the second polyrotaxane comprise, at each of their ends, independently of one another, at least one molecular structure which prevents each of the at least two cyclic molecules and the one linear molecules from separating, wherein the at least one molecular structure is chosen, independently of one another, from molecules and macromolecules.

65. The anhydrous cosmetic composition according to the claim 64, wherein the at least one molecular structure carries such an ionic charge and/or occupies such a volume that it prevents the at least two cyclic molecules and the at least one linear molecule from separating.

66. The anhydrous cosmetic composition according to claim 64, wherein the at least one molecular structure is chosen from:
dinitrophenyl groups;
cyclodextrins;
adamantane groups;
trityl groups;
fluoresceins;
pyrenes; and
naphthalimides.

67. The anhydrous cosmetic composition according to claim 66, wherein the dinitrophenyl groups are chosen from 2,4-dinitrophenyl groups and 3,5-dinitrophenyl groups.

68. The anhydrous cosmetic composition according to claim 37, wherein at least one cyclic molecule of the at least one first polyrotaxane and at least one cyclic molecule of the at least one second polyrotaxane are bonded via at least one bond chosen from chemical bonds and physical bonds, wherein said at least one bond is obtained by reaction of the at least one cyclic molecule of the at least one first polyrotaxane and at least one cyclic molecule of the at least one second polyrotaxane with at least one agent chosen from crosslinking agents, coupling agents, and photocrosslinking agents.

69. The anhydrous cosmetic composition according to claim 68, wherein the crosslinking agents are chosen from cyanuryl chloride, trimesoyl chloride, terephthaloyl chloride, epichlorohydrin, dibromobenzene, glutaraldehyde, phenylene diisocyanates, tolylene diisocyanates, 1,1′-carbonyldiimidazole, divinyl sulphone, acid dichlorides, acids substituted by a trichloro group, and alkoxysilanes.

70. The cosmetic product according to claim 69, wherein the crosslinking agent is tolylene 2,4-diisocyanate.

71. The cosmetic product according to claim 69, wherein the crosslinking agent is sebacoyl dichloride.

72. The anhydrous cosmetic composition according to claim 69, wherein the crosslinking agents are chosen from cyanuryl chloride, tolylene 2,4-diisocyanate, 1,1′-carbonyldiimidazole, trimesoyl chloride, terephthaloyl chloride, and alkoxysilanes.

73. The anhydrous cosmetic composition according to claim 72, wherein the alkoxysilanes are chosen from tetramethoxysilane and tetraethoxysilane.

74. The anhydrous cosmetic composition according to claim 68, wherein in the coupling agents are chosen from silane coupling agents and titanium coupling agents.

75. The anhydrous cosmetic composition according to claim 74, wherein in the silane coupling agents are chosen from alkoxysilanes and the titanium coupling agents are chosen from alkoxytitanium coupling agents.

76. The anhydrous cosmetic composition according to claim 68, wherein the photocrosslinking agent is chosen from stilbazolium salt based photocrosslinking agents.

77. The anhydrous cosmetic composition according to claim 76, wherein the stilbazolium salt based photocrosslinking agents are chosen from formylstyrylpyridinium salts, cinnamic acid salts, anthracene salts, and thymine salts.

78. The anhydrous cosmetic composition according to claim 36, wherein the at least one oil is chosen from silicone oils and hydrocarbon oils.

79. The anhydrous cosmetic composition according to claim 78, wherein the at least one oil is chosen from volatile oils and non-volatile oils.

81. The anhydrous cosmetic composition according to claim 79, wherein the at least one oil is chosen from volatile hydrocarbon oils.

82. The anhydrous cosmetic composition according to claim 81, wherein the volatile hydrocarbon oils are chosen from hydrocarbon oils having from 8 to 16 carbon atoms.

83. The anhydrous cosmetic composition according to claim 82, wherein the hydrocarbon oils having from 8 to 16 carbon atoms are chosen from branched C₈-C₁₆ alkanes.

83. The anhydrous cosmetic composition according to claim 82, wherein the branched C₈-C₁₆ alkanes are chosen from isododecane, isodecane, and isohexadecane.

84. The anhydrous cosmetic composition according to claim 78, wherein the at least one oil is chosen from volatile silicones, optionally comprising at least one group chosen from alkyl groups having from 1 to 10 carbon atoms and alkoxy groups having from 1 to 10 carbon atoms.

85. The anhydrous cosmetic composition according to claim 84, wherein the volatile silicones are chosen from volatile linear silicone oils having a viscosity of ≦8 centistokes (8×10⁻⁶ m²/s) and from 2 to 10 silicon atoms and cyclic silicone oils having a viscosity of ≦8 centistokes (8×10⁻⁶ m²/s) and from 2 to 10 silicon atoms.

86. The anhydrous cosmetic composition according to claim 84, wherein the volatile silicones comprise from 2 to 7 silicon atoms.

87. The anhydrous cosmetic composition according to claim 36, wherein the at least one oil is chosen from the following non-volatile oils:
hydrocarbon oils of animal origin;
hydrocarbon oils of vegetable origin;
synthetic ethers having from 10 to 40 carbon atoms;
linear hydrocarbons of mineral origin;
linear hydrocarbons of synthetic origin;
branched hydrocarbons of mineral origin;
branched hydrocarbons of synthetic origin;
synthetic esters;
esters of polyols;
esters of pentaerythritol;
esters of dimer diols;
esters of dimer diacids;
fatty alcohols that are liquid at ambient temperature comprising at least one carbon chain having from 12 to 26 carbon atoms chosen from branched carbon chains and unsaturated carbons chains;
higher fatty acids; and
dialkyl carbonates, wherein the 2 alkyl chains may be identical or different.

88. The anhydrous cosmetic composition according to claim 87, wherein the hydrocarbon oils of vegetable origin are chosen from phytostearyl esters.

89. The anhydrous cosmetic composition according to claim 88, wherein the phytostearyl esters are chosen from phytostearyl oleates, phytostearyl isostearates, and lauroyl/octyldodecyl/phytostearyl glutamates.

90. The anhydrous cosmetic composition according to claim 87, wherein the linear hydrocarbons of mineral origin, linear hydrocarbons of synthetic origin, branched hydrocarbons of mineral origin, and branched hydrocarbons of synthetic origin are chosen from liquid petroleum, polydecenes, hydrogenated polyisobutene, and squalane.

91. The anhydrous cosmetic composition according to claim 87, wherein the synthetic esters are chosen from oils of formula R₁COOR₂, wherein
R₁ represents a residue of a linear or branched fatty acid comprising from 1 to 40 carbon atoms, and
R₂ represents a hydrocarbon chain.

92. The anhydrous cosmetic composition according to claim 92, wherein R₂ is chosen from branched hydrocarbon chains comprising from 1 to 40 carbon atoms, provided that R₁+R₂≧10.

93. The anhydrous cosmetic composition according to claim 92, wherein R₂ is chosen from cetostearyl octanoate, esters of isopropyl alcohol, ethyl palmitate, 2-ethylhexyl palmitate, isopropyl stearate, isopropyl isostearate, aryl isostearate, octyl stearate, hydroxylated esters, ricinoleates of alcohols, ricinoleates of polyalcohols, C₁₂ to C₁₅ alcohol benzoates, hexyl laurate, esters of neopentanoic acid, esters of isononanoic acid, hydroxylated esters, esters of polyols, esters of pentaerythritol, esters of dimer diols, esters of dimer diacids, fatty alcohols which are liquid at ambient temperature comprising at least one carbon chain having from 12 to 26 carbon atoms chosen from branched carbon chains and unsaturated carbons chains, and higher fatty acids.

94. The anhydrous cosmetic composition according to claim 36, wherein the at least one oil is chosen from:
non-volatile silicone oils;
phenylated silicones;
dimethicones;
phenyl(trimethylsiloxy)diphenylsiloxanes, diphenyl dimethicones, diphenyl(methyldiphenyl)trisiloxanes, (2-phenylethyl)trimethylsiloxysilicates with a viscosity of less than or equal to 100 cSt, dimethicones with a viscosity of less than or equal to 100 cSt, and phenyl trimethylicones with a viscosity of less than or equal to 100 cSt.

95. The anhydrous cosmetic composition according to claim 94, wherein the non-volatile silicone oils are chosen from:
non-volatile polydimethylsiloxanes;
non-volatile polydimethylsiloxanes comprising groups chosen from pendent alkyl groups, pendent alkoxy groups, and C₂-C₂₄ alkyl groups at the ends of the silicone chain; and
phenylated silicones.

96. The anhydrous cosmetic composition according to claim 95, wherein the phenylated silicones are chosen from phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxysilicates with a viscosity of less than or equal to 100 cSt, dimethicones with a viscosity of less than or equal to 100 cSt, and phenyl trimethylicones with a viscosity of less than or equal to 100 cSt.

97. The anhydrous cosmetic composition according to claim 37, further comprising at least one coloring material.

98. The anhydrous cosmetic composition according to claim 38, wherein the at least one coloring material is chosen from dyes, pigments and lakes.

99. The anhydrous cosmetic composition according to claim 37, further comprising at least one component chosen from waxes, pasty fatty substances, and fillers.

100. The anhydrous cosmetic composition according to claim 37, wherein it is in a form chosen from a product for making up at least one of lips, eyelids, eyebrows and skin and a product for caring for at least one of lips, eyelids, eyebrows and skin.

101. The anhydrous cosmetic composition according to claim 37, wherein at least one cyclic molecule is α-cyclodextrin and at least one linear molecule is a polyethylene glycol.

102. The anhydrous cosmetic composition according to claim 36, wherein the content of the crosslinked polyrotaxane ranges from 0.1 to 80% by weight, with respect to the total weight of the composition.

103. The anhydrous cosmetic composition according to claim 102, wherein the content of the crosslinked polyrotaxane ranges from 1 to 30% by weight with respect to the total weight of the composition.

104. The anhydrous cosmetic composition according to claim 103, wherein the content of the crosslinked polyrotaxane ranges from 3 to 25% by weight, with respect to the total weight of the composition.

105. A process for caring for and/or making up keratinous substances other than the hair, comprising applying to the keratinous substances an anhydrous cosmetic composition comprising at least one crosslinked polyrotaxane and at least one oil.
